(19) United States
(12) Patent Application Publication
(10) Pub. No.: US 2011/0060126 A1
(43) Pub. Date: Mar. 10, 2011

(54) METHOD AND APPARATUS FOR OPTIMIZING CRYSTALLIZATION CONDITIONS OF A SUBSTRATE

(76) Inventors: Jean-Pascal Viola, Rockville, MD (US); Christian Houde, Montreal (CA); Steven Tetreault, Saint Hubert (CA)

(21) Appl. No.: 11/572,191

(22) PCT Filed: Jul. 15, 2005
(86) PCT No.: PCT/CA05/01118

§ 371 Date: Nov. 24, 2010

Related U.S. Application Data
(60) Provisional application No. 60/588,359, filed on Jul. 16, 2004.

(51) Int. Cl. C07K 14/00; B01J 19/00

(52) U.S. Cl. 530/350; 422/129

(57) ABSTRACT

The present invention relates to a multi-well crystallization plate comprising a plurality of wells, each well having therein a different crystallization media. Each crystallization media varying according to at least two different parameters. The first parameter has at least one condition, and the second parameter has at least two different conditions, whereby the multi-well plate allows facilitating optimization of crystallization conditions of a substrate. Methods for optimizing crystallization conditions of a substrate are also disclosed.

### TECHNICAL FIELD

The present invention relates to improvements in the field of crystallography. In particular, this invention relates to a new method or strategy for optimizing crystallization conditions of a given substrate. The invention also relates to a new multi-well plate for carrying out the optimization of the crystallization conditions of the substrate.

### BACKGROUND OF THE INVENTION

During the last decade, the technical aspect of structural biology has been greatly simplified by high-throughput methods, applied from protein expression up to data collection. Even with that gained advantage, Crystal Growth remains an important challenging step of crystallography. As automation processes are becoming routine in laboratories, increasing the number of performed crystallization experiments on a day to day basis, there is still a constant decrease in the success value of these experiments (# Structure solved/Experimental Setup).

In order to obtain crystals for protein 3D structure determination, crystallographers use well known strategies where a protein is initially screened against a wide array of conditions in order to determine a “hit solution” for a given protein. From this set of initial conditions, crystalline forms or “hits” are observed and several optimization rounds, centered on the initial condition producing the hit, are often necessary to get essential quality crystal.

The most popular and used optimization strategy is performed by varying components of the experimental chemical conditions and preparing grids around the initial hit (for a clear review, see McPherson, A. Crystallization of biological macromolecules. 1999. New-York: Cold Spring Harbor Library Press, 291-296). This approach allows to determine which factor influences crystallization of a particular protein and to what extent it can improve crystal quality. Many parameters can be varied in trying to optimize an initial hit. Such parameters are, for example, precipitant concentration, pH, type of buffer, salt ions, additives such as reducing agents, metal ions, inhibitors etc., protein (concentration, source, mutant etc.), and experimental conditions (temperature, methods, etc.).

However, when working with a new protein, even a very experienced crystallographer may have some difficulties selecting which factors are important and which are not.

The method using the “expanded grid” is a very well designed strategy of optimization but it constitutes a tedious and time consuming procedure. Also, rounds of optimization centered on an initial crystallization hit does not always bring the ultimate goal of getting a crystal since the hit may itself be the optimized condition corresponding to this particular chemical environment.

Optimization of crystallization condition is usually carried out by slight variations of the chemical environment around an initial hit. Such a process is tedious and time consuming since many questions must be asked in order to determine which factors must be varied first, how to apply the selected changes to initial hit and in what format. The crystallographer also has to determine if the variations brought to the parameters significantly vary initial crystallization hit and create a new condition and if the crystallization space around the hit is well covered. Therefore, it appears that the methods and strategies proposed so far do not provide efficient and rapid solution for the optimization of the crystallization conditions of a protein, and that new methods would be required.

Macromolecular crystallization keeps getting faster and easier to setup, but crystal growth still remains a trial & error process. It is rare that an initial screening alone provides high-resolution crystals. Many rounds of optimization are necessary to get diffraction quality crystals.

With automation and high-throughput techniques present in more and more laboratories, “mild results” in initial screenings of protein alone still points toward the fact that the methodology aspect of crystal growth needs a second look. In particular, the relationship between initial screening and optimization requires more attention.

### SUMMARY OF THE INVENTION

It is therefore an object of the present invention to provide a method and an apparatus for optimizing crystallization conditions, which would overcome the above-mentioned drawbacks.

It is another object of the present invention to provide a method and an apparatus for rapidly and simply optimizing crystallization conditions of a substrate.

It is another object of the present invention to provide a method and for optimizing crystallization conditions of a substrate, which could be carried out in a single round after the determination of the hit solution.

According to one aspect of the invention, there is provided a multi-well plate comprising a plurality of wells, each well having therein a different crystallization media, each crystallization media varying according to at least two different parameters, a first parameter having at least one condition, and a second parameter having at least two different conditions, whereby said multi-well plate allows to facilitate optimization of crystallization conditions of a substrate.

The parameters may be for example selected from the group consisting of a buffer, pH of said crystallization media, salt, concentration of said salt, temperature of said crystallization media, additive, concentration of said additive, co-crystallization compound, concentration of said co-crystallization compound, alcohol, concentration of said alcohol, polymer, and concentration of said polymer.

In one embodiment of the invention, one of said parameters is the buffer. Each condition of said buffer parameter can be represented by a predetermined buffer that can be selected from the group consisting of Tris, Tris HCI, HEPES, Sodium HEPES, Imidazole, Sodium Citrate, Sodium Cacodylate and Sodium Acetate.

Alternatively, one of said parameters can be the pH of said crystallization media. Each condition of the pH can represents a different pH value to be tested.

One of said parameters can also be the salt. Each condition of the salt can thus represents a different salt, that can each comprise an inorganic or an organic anion, and an organic cation, or alternatively, an organic anion, and an inorganic or an organic cation.

The cation can be for example selected from the group consisting of sodium, potassium, ammonium, magnesium, calcium and lithium, and the anion can be selected from the group consisting of formate, malonate, chloride, acetate, fluoride, bromide, nitrate and thiocyanate.

In one embodiment, one of said parameters is the concentration of the salt. Each condition of the salt concentration can thus be represented by a different concentration value of said salt.

In another embodiment of the invention, one of said parameters is the temperature of said crystallization media, where each condition of the temperature media can thus be represented by a different temperature to be tested.

In a further embodiment of the invention, one of said parameters is the additive, and thus each condition of the additive can be represented by a different additive, such as a reducing agent, a metal ion, an inhibitor or a detergent.

Still in one embodiment of the invention, one of said parameters is the concentration of said additive, where each condition of the additive concentration can thus be represented by a different concentration value of said additive to be tested.

In a further embodiment of the invention, one of said parameters is the ligand, where each condition of the ligand can thus be represented by a different ligand to be tested. For example, the predetermined ligand can be selected from the group consisting of ATP, ADT, NAD, NADP, NADPH, NADH.

In a further embodiment of the invention, one of the parameters is the concentration of the ligand, where each condition of the ligand concentration can thus be represented by a different concentration value of said ligand to be tested.

In a further embodiment of the invention, one of the parameters is the alcohol, where each condition of the alcohol can be represented by a predetermined alcohol to be tested. Examples of alcohol can be selected from the group consisting of methanol, ethanol, propanol isopropanol, methylpentanediol, hexanediol, and ethylene glycol.

In a further embodiment of the invention, one of the parameters is the concentration of said alcohol, where each condition of the alcohol concentration to be tested can thus be represented by a different concentration value of said alcohol.

In a further embodiment of the invention, one of the parameters is the polymer, where each condition of the polymer can thus be represented by a different polymer to be tested, such as PEG, polyethyleneimine and Jeffamine M-600.

In a further embodiment of the invention, one of the parameters is the concentration of said polymer, where each condition of the polymer concentration to be tested can thus be represented by a different concentration value of said polymer.

The crystallization media can thus vary according to at least two, preferably more than two and more preferably three different parameters, where a first parameter has at least one condition, and a second parameter has at least two different conditions, and a third parameter has at least one and preferably two, condition.

In another embodiment of the invention, the first parameter is the additive, said second parameter is the concentration of said additive, and said third parameter is the pH of said crystallization media. In still a further embodiment of the invention, the first parameter is the salt, said second parameter is the concentration of said salt, and said third parameter is the pH of said crystallization media.

The plate is a multi-well plate that can comprise any number of wells such as 3, 6, 24, 96, 192, 384, 768 or 1536 wells, and more preferably 96 wells.

In yet a further embodiment of the invention, there is provided a plate as defined above and comprising 96 wells, said first parameter being the salt and the conditions of said first parameter being 16 different salts, said second parameter being the salt concentration and the conditions of said second parameter being 2 different concentrations, and said third parameter being the pH and the conditions of said third parameter are 3 different pH values.

The crystallization media used in the plate can either be a solution or a gel. The plate preferably further comprises a cover disposed on said wells to seal them.

The plate can be of the hanging-drop crystallization type of plate, the plate further comprising a cover for sealing said wells, or of the sitting drop crystallization type of plate.

Each well of the plate may comprise a crystallization media reservoir adjacent to a substrate well.

The plate can be used to crystallize any crystallisable molecule such as a protein or some organic compounds. The volume of the crystallization media to be used with the plate of the present invention will vary, but generally will be of at least 1 μL, more preferably about 5 to about 500 μL, and most preferably 10 μL of said crystallization media. Preferably, the crystallization media is contained in a crystallization media reservoir to the substrate well.

In accordance with the present invention, there is also provided a method for optimizing crystallization conditions for a substrate comprising the step of adding said substrate into each well of a plate as defined above.

The method may additionally further comprise adding a hit solution for said substrate in each well before or after adding said substrate in each well.

Further in accordance with the present invention, there is provided a method for optimizing crystallization conditions for a substrate comprising the step of contacting said substrate with a hit solution for said substrate, and said crystallization media into each well of a plate as defined above.

Still in accordance with the present invention, there is provided a method for optimizing crystallization of a substrate comprising:
a) determining a hit solution for said substrate by screening different solutions;
b) adding said hit solution determined in step a) into each media reservoir of a plate as defined above so as to obtain a mixture;
c) adding a substrate into substrate wells;
d) transferring a desired volume of said mixture from each media reservoir to the substrate wells; and
e) sealing said substrate wells and media reservoir and allowing for crystallization of the substrate.

In a further embodiment of the present invention, there is also provided a method for optimizing crystallization conditions for a substrate comprising:
a) determining a hit solution for said substrate by screening different solutions;
b) adding said hit solution into a media reservoir of a plate as defined above so as to obtain a mixture of said crystallization media and said hit solution;
c) adding said substrate in the substrate well;
d) adding the mixture obtained at step b) in the substrate well; and
e) sealing the media reservoir and the adjacent substrate well with said cover, and allowing crystallization of the substrate.

Applicant has found that by using the above-mentioned plate or methods, it is possible to rapidly optimize the crystallization conditions for a given substrate. Moreover, when using such a plate or methods, it is possible to rapidly obtain considerable amount of information concerning optimal conditions for a given substrate. This plate or these methods permit to directly use an initial hit solution, hereby improving the reproducibility, and straightforward analysis. Moreover, this plate or these methods permit a wider coverage of the crystallization space. It is also possible to carry out a direct testing of concentration, pH variation and additives effect on crystallization.

In the plate or methods of the invention, the parameters can be selected from the group consisting of a buffer, pH of the crystallization media, salt, concentration of the salt, temperature of the crystallization media, additive, concentration of the additive, ligand (or co-crystallization compound), concentration of the ligand, alcohol, concentration of the alcohol, polymer, concentration of the polymer.

The method and apparatus of the invention are useful for optimizing crystallization conditions of substrates such as proteins.

### DESCRIPTION OF PREFERRED EMBODIMENTS

Further features and advantages of the invention will become more readily apparent from the following description of preferred embodiments as illustrated by way of examples in the appended drawings wherein:

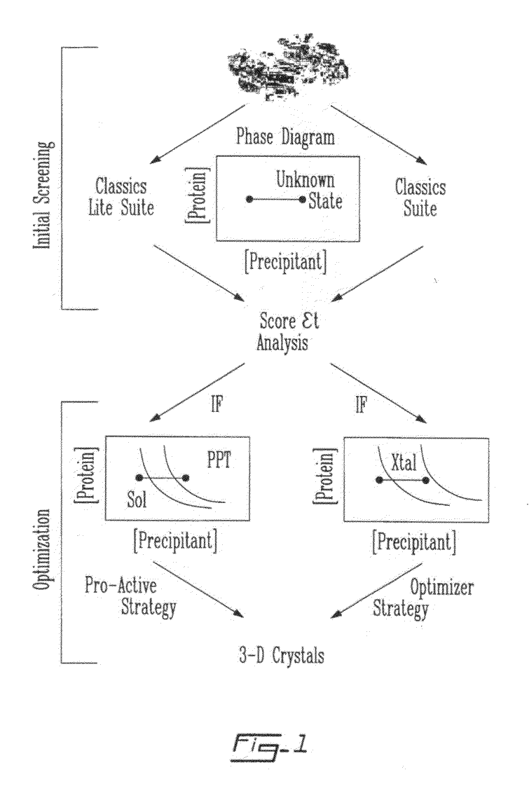

FIG. 1 illustrates the integration of an initial screening with an optimization step;

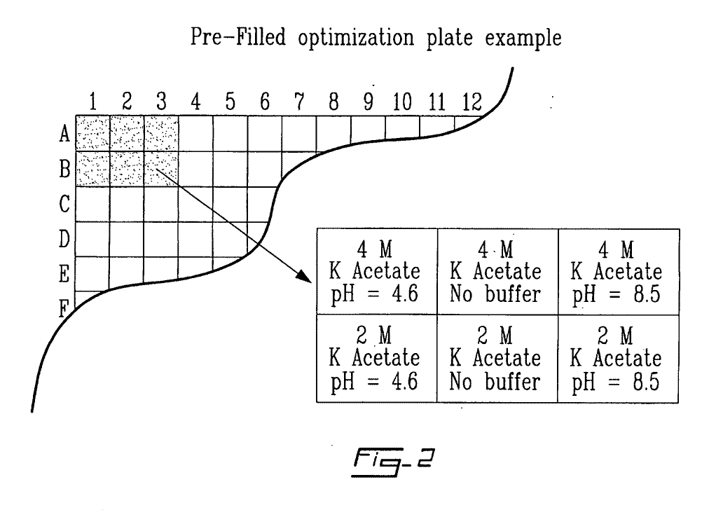

FIG. 2 is a schematic view of a crystallization plate according to a preferred embodiment of the invention;

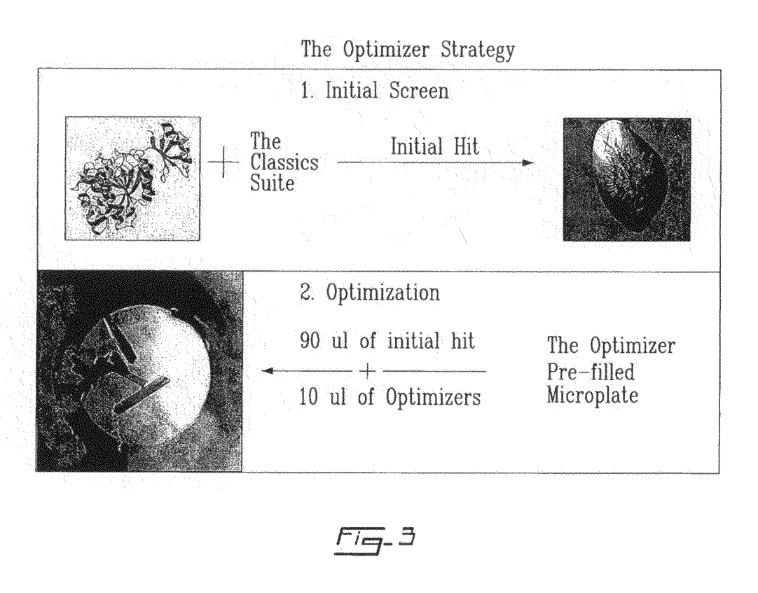

FIG. 3 is a flow chart diagram illustrating a method according to another preferred embodiment of the invention;

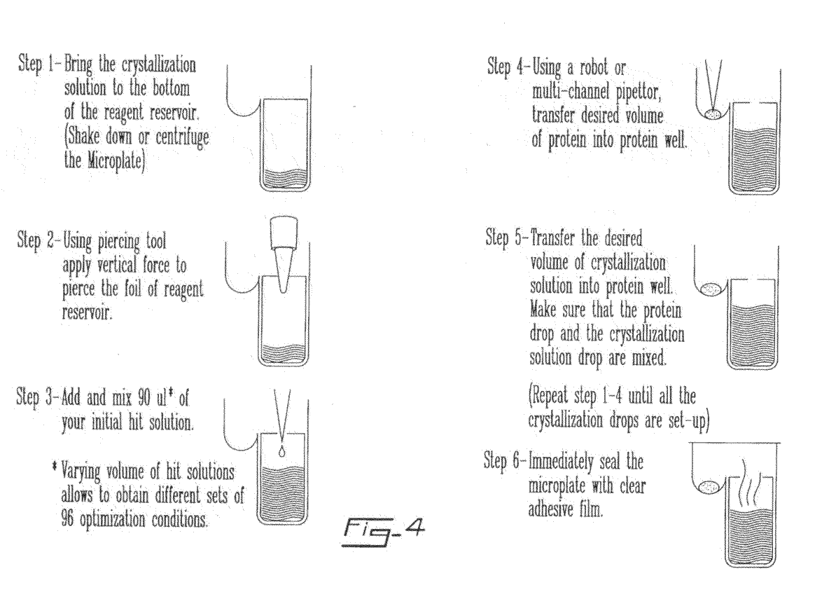

FIG. 4 is flow chart diagram illustrating a method according to another preferred embodiment of the invention;

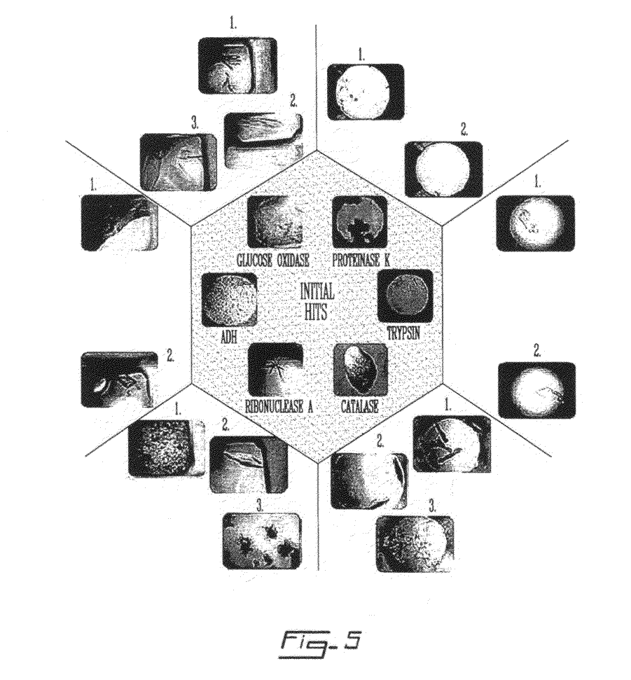

FIG. 5 illustrates results obtained with the optimizer plate on six different proteins;

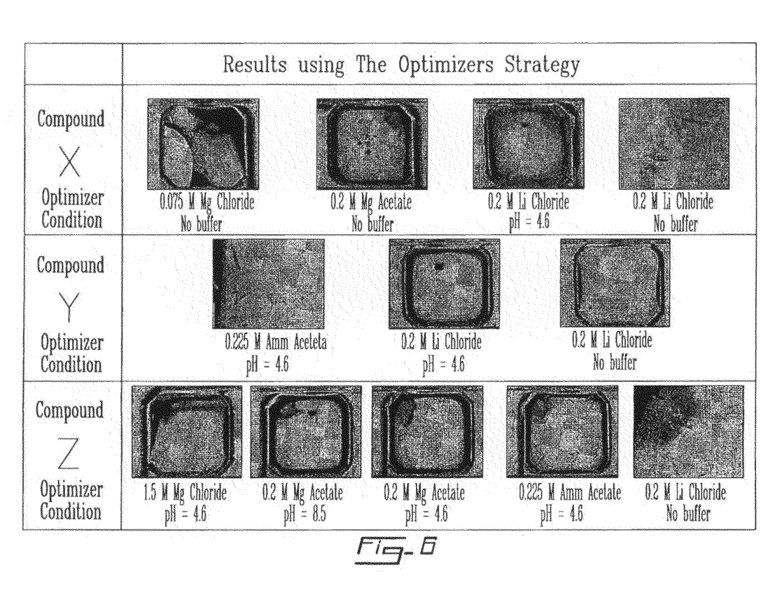

FIG. 6 is diagram showing results obtained after using a crystallization plate and a method according to another preferred embodiment of the invention; and

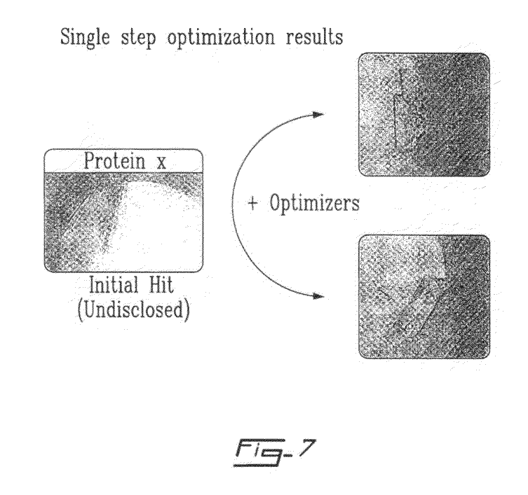

FIG. 7 is flow chart diagram showing results obtained after using a crystallization plate and a method according to another preferred embodiment of the invention.

### DETAILED DESCRIPTION OF THE PREFERRED EMBODIMENT

In accordance with one embodiment of the invention, there is presented herewith a strategy which takes advantage of a closer connection between 2 elements of a successful crystal growth experiment: Initial screening and Optimization. The proposed strategy combines a variation in the original initial screening and a subtle change in its analysis.

Initial screening is combined with optimization to minimize time and protein use, while maximizing success. This is however not done easily since one of the problems is the biased and incomplete analysis of the initial screen results. it is biased since i) classification and optimization is only performed around observable crystal forms, and ii) all drops not showing a crystal form are scored and kept aside.

Usually, initial screens gives “Initial hits”. If lucky, these hits will contain high resolution crystals and the protein structure will be solved easily. But it is rarely the case. In most case, one can expect to obtain:
a “Good-hit” where crystal forms are present, and can be optimized easily/directly around the crystallization condition to produce the crystal wanted;
a “Bad-hit” where crystal forms are present, but that are hard or impossible to optimize directly into anything else better; and
a “Missed-hit” whose initial result showed precipitation or remained clear.

If “Missed-Hits” are not paid attention to, the “Best-hit” may be missed altogether simply because something else than a crystal form was seen in the initial screening.

The present invention thus allows maximizing success by improving initial screening results analysis to select the optimization technique.

Presented herein in accordance with the present invention is a new method where the selection of the crystallization solution and experiment scoring in initial screening strategy are modified to get more information on protein solubility behavior. An analysis of the results, paying close attention to those “Missed-Hits”, guides the crystallographer toward the proper optimization strategies to use next. Essentially the method comprises the steps of:
Preparing initial screens such as Classics and Classics Lite (Anions, Cations, pHClear I and II can also be used) where each condition is duplicated at half the precipitant concentration to get 2 data points on each unique phase diagram.
Differential analysis of results where a comparison of the precipitant concentration is now available, and where with 2 data points present, the information is greatly increased; and
Selecting an optimization strategy using the optimizer plate of the present invention.

Using this crystallization strategy, less protein is required, which allows for more analysis, less time is also required to obtain a best hit from a protein in solution to an X-ray quality crystal, and consequently, money is saved by using less protein and taking less time.

The integration of an initial screening with an optimization step as in the method of the present invention is illustrated in FIG. 1. Illustrated in FIG. 1 is the integration between the initial screening and optimization. First, a protein such as a commercially available protein is prepared as is currently done in the art. The protein preparation is then dialyzed, and any necessary additives are added. Then, an initial screening strategy of 2 identical conditions was used, where the only difference is having the main precipitant at a 1× (Classic or standard) and 0.5× concentration (Classic lite). This allows a direct comparison in the phase diagram, where initially it is not known under what phase the protein will be found in each condition. The results of these screenings are then analyzed and scored according to whether crystalline forms, precipitation (either granulous or amorphous) or clear forms are obtained. The results are analyzed side by side for each condition used and the drops are compared. Finally, the best result obtained is then subjected to optimization on the optimizer plate to obtain 3-D crystals.

In accordance with a preferred embodiment of the present invention, there is also provided a new plate was developed to facilitate and accelerate optimization set up while respecting experimental constraints. This new plate will be called, hereinafter, the Optimizer plate. Such a plate comprises:
96 well crystallization plate (available in several different formats from Corning and Greiner). The wells are pre-filled with a 10 μl aliquot of 96 optimization solutions (crystallization media).
As presented below in a particular experiment and in FIG. 2, these solutions (crystallization media) may comprise 16 chemical solutions at 2 concentrations (2 and 4M) and 3 different pH (no buffer, 4.6 and 8.5), each chemical solution being displayed in a mini-grid. Table 1 summarizes the parameters and conditions of one of the mini grid of FIG. 2.

TABLE 1

| Parameters | 1^{st} condition | 2^{nd} condition | 3^{rd} condition |
|---|---|---|---|
| # 1 | potassium acetate | — | — |
| Salt | | | |
| #2 | 2M | 4M | — |
| Salt concentration | | | |
| # 3 | no buffer | pH = 4.6 | pH = 8.5 |
| buffer (pH) | | | |

It has been found that by simply adding 90 μL of an initial hit solution (following an initial screen—see FIG. 3.) to each reservoir (or crystallization media reservoir) of the 96 pre-filled wells, 96 new optimization crystallization conditions can be prepared in minutes (see FIG. 4). In FIG. 4, the substrate well and the crystallization media (or solution) can be seen. In. FIG. 4, 6 different steps in accordance with one embodiment of the invention are illustrated. Briefly, the crystallization solution is added to the bottom of the reagent reservoir. If need be, the plate can be shaken down or centrifuged. Then a piercing tool is used to pierce or break the foil of the reagent reservoir using force. 90 μl of the initial hit solution is then added to the crystallization solution. Varying volumes of hit solutions allows obtaining different sets of 96 optimization conditions. Using a robot or a multi-channel pipettor, a desired volume of protein to be crystallized is transferred into the protein well. Then the desired volume of crystallization solution is transferred into the protein well and is mixed with the protein drop. The above can be repeated until all the crystallization drops are set up. Finally, the microplate is sealed with clear adhesive film.

The principal advantages of this Pre-Filled optimization plate are:
Fast and easy, for manual or automatic setups (minutes);
Combined grid and additive approach;
Direct use of initial hit solution (improved reproducibility);
Straightforward analysis;
Wider coverage of the crystallization space; and
Direct testing of concentration, pH variation and additives effect on crystallization.

It has been shown in table 2, that when using a pre-filled optimizer plate, clear improvement of crystalline form quality can be observed, more suitable crystals are obtained, and different crystal forms for the same protein can be also obtained. The set-up is much simpler and faster and the “time-to-crystal” is reduced.

Of course, one skilled in the art will appreciate that the method and Optimizer plate of the present invention can make use of more different conditions, so as to fill up a plate.

TABLE 2
Comparison of optimization results using usual strategy and the
optimizer of the present invention

| | Hit | Initial Results | | |
|---|---|---|---|---|
| Protein | Solution | from The Classics | Usual optimization | The Optimizers |
| Catalase | NCL-37 | Needles | Needles - Precipitate | Large 3D crystals |
| | NCL-53 | Needles - Precipitate | Needles - Precipitate | Large 3D crystals |
| | NCL-64 | Needles - Precipitate | Needles - Precipitate | Large 3D crystals |
| A-Lactalbumin | NCL-34 | Needles - Precipitate | Small 3D crystals | Large 3D crystals |
| | NCL-74 | Microcrystals | Precipitate | Large 3D crystals |
| Pepsin | NCL-44 | Precipitate | Precipitate | Small 2D crystals |
| Ribonuclease A | NCL-90 | Microcrystals | Needles - Precipitate | Small 3D crystals |
| Thaumatin | NCL-22 | Microcrystals | Small 3D crystals | Large 3D crystals |

The Mini-grid optimization approach (see FIG. 2) allows crystallographers to evaluate the relative importance of the different factors such as chemical species of the additive, concentration, and pH.

From table 3, it can be seen that, depending on the protein to crystallize and the initial condition, different optimization components show different influences, demonstrating the importance of a wider sampling of crystallization space in optimization strategies.

TABLE 3
Relative important of the optimizer components
for optimized protein crystals

| | | | Preliminary |
|---|---|---|---|
| Protein | Optimizer | pH | Conclusions |
| Catalase | 0.32 M | 4.6, No, 8.5 | Sodium Chloride is |
| NCL-37 | Sodium Chloride | | key pH seems to have |
| | 0.16 M | 4.6, 8.5 | little effect |
| | Sodium Chloride | | |
| Catalase | 0.2 M | 4.6 | Low pH is key |
| NCL-53 | Magnesium acetate | | |
| | 0.11 M | 4.6 | |
| | Potassium chloride | | |
| Catalase | 0.4 M | 8.5 | No precise factor |
| NCL-64 | Potassium acetate | | identified |
| | 0.24 M | No | |
| | Sodium malonate | | |
| | 0.11 M | 8.5 | |
| | Potassium chloride | | |
| | 0.12 M | No | |
| | Sodium thiocyanate | | |
| | 0.175 M | 4.6, No | |
| | Sodium nitrate | | |
| α- | 0.35 M | 8.5 | High pH is key |
| Lactalbumin | Sodium bromide | | Salt identify seems to |
| NCL-34 | 0.1 M | No, 8.5 | have little importance |
| | Magnesium acetate | | |
| | 0.06 M | No, 8.5 | |
| | Sodium fluoride | | |
| | 0.03 M | 4.6, 8.5 | |
| | Sodium fluoride | | |
| | 0.12 M | No, 8.5 | |
| | Sodium thiocyanate | | |
| Pepsin | 0.11 M | 4.6 | Highly specific |
| NCL-44 | Calcium chloride | | condition needed |
| Thaumatin | 0.2 M | 8.5 | Potassium seems to be |
| NCL-22 | Potassium acetate | | necessary |
| | 0.22 M | 8.5 | |
| | Potassium chloride | | |
| | 0.11 M | No | |
| | Potassium chloride | | |

FIG. 5 illustrates results obtained with the optimizer plate on six different proteins. In the center of the hexagon, typical initial hits are shown for the six proteins displayed therein. As can be seen, none of the drops in the center of the figure shows any 3-D crystals which can be used with X-ray. In the six regions of the hexagon are examples of the results obtained with the Optimizer plate of the present invention using a single plate. The experimental information is presented in Table 4.

TABLE 4
Experimental conditions used

| Protein | Initial Hit | Optimizer Hit |
|---|---|---|
| Glucose Oxidase | NCL-81 | #1 - 3.5M Na Bromide |
| | Salt: — | #2 - 1.6M Na Chloride and |
| | Buffer: — | 0.1M Na Acetate (pH 4.6) |
| | Ppt: 30% (W/v) PEG 1500 | |
| | | #3 - 4M Li Chloride |
| Proteinase K | NCL-62 | #1 - 1.0M Tris-HCl (pH 8.5) |
| | Salt: — | #2 - 2.25M Ammonium Acetate |
| | Buffer: 0.1M MES pH 5.6 | |
| | Ppt: 1.6M Mg Sulphate | |
| Trypsin | NCL-28 | #1 - 2.25M Ammonium Acetate |
| | Salt: — | |
| | Buffer: 0.1M HEPES pH 7.5 | #2 - 1.2M Na Malonate and |
| | Ppt: 2M Ammonium Formate | 0.1M Tris-HCl (pH 8.5) |
| Catalase | NCL-27 | #1 - 3.5M Na Bromide and |
| | Salt: — | 0.1M Tris-HCl pH 8.5 |
| | Buffer: 0.1M Tris-HCl pH 8.5 | #2 - 1.2M Na Citrate and |
| | Ppt: 2M Ammonium Sulfate | 0.1M HEPES pH 7.5 |
| | | #3 - 65% (v/v) MPD and |
| | | 0.1M MES (pH 6.0) |
| Ribonuclease A | NCL-82 | #1 - 1.1M Ca Chloride |
| | Salt: 0.01M Ni Chloride | #2 - 4M Li Chloride |
| | Buffer: 0.1M Tris pH 8.5 | #3 - 2M Li Chloride |
| | Ppt: 20% (w/v) PEG 2000 MME | |
| Alcohol | NCL-54 | #1 - 1.2 Na Malonate |
| dehydrogenase | Salt: 0.2M Na Chloride | #2 - 1.2M Malonate and |
| | Buffer: 0.1M HEPES pH 7.5 | 0.1M Na Acetate (pH 4.6) |
| | Ppt: 2M Ammonium Sulfate | |

Initial hit: a description of the hit condition is given;
NCL refers to the Classic Suite and NTL is for the Classic Lite Suite;
Optimizer added: initial conditions of the optimizer used to create the winning condition are given; for example, 10 μl of the condition given for the Optimizer is mixed with 90 μl of the initial hit condition in a pre-filled microplate.

Table 5 provides for a summary of the results obtained with the conditions of Table 4 and illustrated in FIG. 5.

TABLE 5
Results of integration of the initial screening and
optimization selection

Crystal Forms

| | Presence of | Optimizer |
|---|---|---|
| Protein | initial hits | Success |
| Alcohol | Yes | Yes |
| Dehydrogenase | | |
| Glucose Oxydase | Yes | Yes |
| Proteinase K | Yes | Yes |
| Ribonuclease A | Yes | Yes |
| Trypsin | Yes | Yes |
| Catalase | Yes | Yes |

According to the results of the initial screening, the optimization step is automatically applied to initial-hit conditions and the presence of improvement in the crystals is shown in FIG. 5.

The present invention will be more readily understood by referring to the following examples which are given to illustrate the invention rather than to limit its scope.

### Example I

### Typical Content of a 96-Well Plate

Table 6 below lists the current content of one of the plate design for optimization of crystallization designed by the Applicant. Of course numerous other modifications could be made, be the example is only being given for illustrative purpose. To be noted that two negative controls have been introduced to confirms results obtained, i.e. well no. 1 and well no. 13. Well no. 1 has been left empty to verify the reproducibility of the assay and well no. 13 was filled with equal volume (compared to the other wells) of water to verify the effects of dilution on the initial parameters. The controls have never been used in such an assay as in initial screening, there is no incentive to leave blank well. Thus one skilled in the art would not be led to create a plate as the one in Table 6, with the two control wells.

TABLE 6
Content of a plate

| Well | | |
|---|---|---|
| number | Content | |
| 1 | | |
| 2 | 0.1 M Sodium Acetate pH 4.6 | ddH₂O |
| 3 | 0.1 M MES 6.5 | ddH₂O |
| 4 | 0.1 M Sodium Acetate pH 4.6 | 3.2 M Sodium chloride |
| 5 | | 3.2 M Sodium chloride |
| 6 | 0.1 M Tris-HCl pH 8.5 | 3.2 M Sodium chloride |
| 7 | 0.1 M Sodium Acetate pH 4.6 | 2.4 M Sodium malonate |
| 8 | | 2.4 M Sodium malonate |
| 9 | 0.1 M Tris-HCl pH 8.5 | 2.4 M Sodium malonate |
| 10 | 0.1 M Sodium Acetate pH 4.6 | 1.5 M Magnesium chloride |
| 11 | | 1.5 M Magnesium chloride |
| 12 | 0.1 M Tris-HCl pH 8.5 | 1.5 M Magnesium chloride |
| 13 | | ddH₂O |
| 14 | 0.1 M HEPES pH 7.5 | ddH₂O |
| 15 | 0.1 M Tris-HCl pH 8.5 | ddH₂O |
| 16 | 0.1 M Sodium Acetate pH 4.6 | 1.6 M Sodium chloride |
| 17 | | 1.6 M Sodium chloride |
| 18 | 0.1 M Tris-HCl pH 8.5 | 1.6 M Sodium chloride |
| 19 | 0.1 M Sodium Acetate pH 4.6 | 1.2 M Sodium malonate |
| 20 | | 1.2 M Sodium malonate |
| 21 | 0.1 M Tris-HCl pH 8.5 | 1.2 M Sodium malonate |
| 22 | 0.1 M Sodium Acetate pH 4.6 | 0.75 Magnesium chloride |
| 23 | | 0.75 Magnesium chloride |
| 24 | 0.1 M Tris-HCl pH 8.5 | 0.75 Magnesium chloride |
| 25 | 0.1 M Sodium Acetate pH 4.6 | 1.2 M Sodium citrate |
| 26 | | 1.2 M Sodium citrate |
| 27 | 0.1 M Tris-HCl pH 8.5 | 1.2 M Sodium citrate |
| 28 | 0.1 M Sodium Acetate pH 4.6 | 2 M Magnesium acetate |
| 29 | | 2 M Magnesium acetate |
| 30 | 0.1 M Tris-HCl pH 8.5 | 2 M Magnesium acetate |
| 31 | 0.1 M Sodium Acetate pH 4.6 | 3.5 M Ammonium chloride |
| 32 | | 3.5 M Ammonium chloride |
| 33 | 0.1 M Tris-HCl pH 8.5 | 3.5 M Ammonium chloride |
| 34 | 0.1 M Sodium Acetate pH 4.6 | 3.5 M Sodium bromide |
| 35 | | 3.5 M Sodium bromide |
| 36 | 0.1 M Tris-HCl pH 8.5 | 3.5 M Sodium bromide |
| 37 | 0.1 M Sodium Acetate pH 4.6 | 0.6 M Sodium citrate |
| 38 | | 0.6 M Sodium citrate |
| 39 | 0.1 M Tris-HCl pH 8.5 | 0.6 M Sodium citrate |
| 40 | 0.1 M Sodium Acetate pH 4.6 | 1 M Magnesium acetate |
| 41 | | 1 M Magnesium acetate |
| 42 | 0.1 M Tris-HCl pH 8.5 | 1 M Magnesium acetate |
| 43 | 0.1 M Sodium Acetate pH 4.6 | 1.75 M Ammonium chloride |
| 44 | | 1.75 M Ammonium chloride |
| 45 | 0.1 M Tris-HCl pH 8.5 | 1.75 M Ammonium chloride |
| 46 | 0.1 M Sodium Acetate pH 4.6 | 1.75 M Sodium bromide |
| 47 | | 1.75 M Sodium bromide |
| 48 | 0.1 M Tris-HCl pH 8.5 | 1.75 M Sodium bromide |
| 49 | 0.1 M Sodium Acetate pH 4.6 | 3.5 M Sodium formate |
| 50 | | 3.5 M Sodium formate |
| 51 | 0.1 M Tris-HCl pH 8.5 | 3.5 M Sodium formate |
| 52 | 0.1 M Sodium Acetate pH 4.6 | 2.2 M Calcium chloride |
| 53 | | 2.2 M Calcium chloride |
| 54 | 0.1 M Tris-HCl pH 8.5 | 2.2 M Calcium chloride |
| 55 | 0.1 M Sodium Acetate pH 4.6 | 4.5 M Ammonium acetate |
| 56 | | 4.5 M Ammonium acetate |
| 57 | 0.1 M Tris-HCl pH 8.5 | 4.5 M Ammonium acetate |
| 58 | 0.1 M Sodium Acetate pH 4.6 | 0.6 M Sodium fluoride |
| 59 | | 0.6 M Sodium fluoride |
| 60 | 0.1 M Tris-HCl pH 8.5 | 0.6 M Sodium fluoride |
| 61 | 0.1 M Sodium Acetate pH 4.6 | 1.75 M Sodium formate |
| 62 | | 1.75 M Sodium formate |
| 63 | 0.1 M Tris-HCl pH 8.5 | 1.75 M Sodium formate |
| 64 | 0.1 M Sodium Acetate pH 4.6 | 1.1 M Calcium chloride |
| 65 | | 1.1 M Calcium chloride |
| 66 | 0.1 M Tris-HCl pH 8.5 | 1.1 M Calcium chloride |
| 67 | 0.1 M Sodium Acetate pH 4.6 | 2.25 M Ammonium acetate |
| 68 | | 2.25 M Ammonium acetate |
| 69 | 0.1 M Tris-HCl pH 8.5 | 2.25 M Ammonium acetate |
| 70 | 0.1 M Sodium Acetate pH 4.6 | 0.3 M Sodium fluoride |
| 71 | | 0.3 M Sodium fluoride |
| 72 | 0.1 M Tris-HCl pH 8.5 | 0.3 M Sodium fluoride |
| 73 | 0.1 M Sodium Acetate pH 4.6 | 2.2 M Potassium chloride |
| 74 | | 2.2 M Potassium chloride |
| 75 | 0.1 M Tris-HCl pH 8.5 | 2.2 M Potassium chloride |
| 76 | 0.1 M Sodium Acetate pH 4.6 | 2.4 M Sodium thiocyanate |
| 77 | | 2.4 M Sodium thiocyanate |
| 78 | 0.1 M Tris-HCl pH 8.5 | 2.4 M Sodium thiocyanate |
| 79 | 0.1 M Sodium Acetate pH 4.6 | 3.5 M Sodium nitrate |
| 80 | | 3.5 M Sodium nitrate |
| 81 | 0.1 M Tris-HCl pH 8.5 | 3.5 M Sodium nitrate |
| 82 | 0.1 M Sodium Acetate pH 4.6 | 4 M Lithium chloride |
| 83 | | 4 M Lithium chloride |
| 84 | 0.1 M Tris-HCl pH 8.5 | 4 M Lithium chloride |
| 85 | 0.1 M Sodium Acetate pH 4.6 | 1.1 M Potassium chloride |
| 86 | | 1.1 M Potassium chloride |
| 87 | 0.1 M Tris-HCl pH 8.5 | 1.1 M Potassium chloride |
| 88 | 0.1 M Sodium Acetate pH 4.6 | 1.2 M Sodium thiocyanate |
| 89 | | 1.2 M Sodium thiocyanate |
| 90 | 0.1 M Tris-HCl pH 8.5 | 1.2 M Sodium thiocyanate |
| 91 | 0.1 M Sodium Acetate pH 4.6 | 1.75 M Sodium nitrate |
| 92 | | 1.75 M Sodium nitrate |
| 93 | 0.1 M Tris-HCl pH 8.5 | 1.75 M Sodium nitrate |
| 94 | 0.1 M Sodium Acetate pH 4.6 | 2 M Lithium chloride |
| 95 | | 2 M Lithium chloride |
| 96 | 0.1 M Tris-HCl pH 8.5 | 2 M Lithium chloride |

### Example II

### Case Study 1—Co-Crystallization Ligand-Protein

In this experiment, pre-filled optimizer plate (Greiner 3 well format) was used to optimize co-crystallization condition between a protein and 3 different compounds. Optimized crystallization condition of the native protein was added and mixed in each well of the pre-filled plate.

Each chemical compound having its own characteristics can interfere with the stability/interaction of the crystallization process, possibly preventing the crystallization in the initial condition. The Optimizer plate allows creating small grids around a successful crystallization condition of a protein and finding a proper condition for co-crystallization between the protein and chemical compounds. Shown in FIG. 6 are the results obtained using the optimizer multi-well plate with ACA04 protein (unknown protein to be crystallized pursuant to a research contract made by the Applicant—the identity and nature of the protein being kept secret to the Applicant) and the 3 chemical compounds. In each case, not only does crystallization occurred, but initial analysis of the crystals quality showed increased diffraction for some. Co-crystals and diffraction pattern have thus been obtained for 3 different compounds using only 1 pre-filled optimizer plate.

### Example III

### Case Study 2—Reduced-Time to Quality Crystal

An initial crystallization hit consisting of very thin, needle crystals, not usable for X-ray diffraction was obtained with The Classics Suite. No improvement was achieved when using usual optimization strategy. As a complementary approach, 90 μL of the initial hit solution (unknown protein to be crystallized pursuant to a research contract made by the Applicant—the identity and nature of the protein being kept secret to the Applicant) was added and mixed in each well of the optimizer multi-well plate (Corning conical flat bottom format) and used for optimization. Two very distinct and large protein crystals grown (see FIG. 7) from solutions containing Sodium Bromide (pH=8.5 or unbalanced) corresponding to well C11 and C12 of the optimizer plate. Using a source for a quick analysis with X-ray, protein crystals diffracted to a resolution of 2.8 Angstroms.

As demonstrated in the above examples, using the crystallization plate of the invention, it has been possible to successfully optimized crystallization conditions for 5 commercially available proteins. Starting with needles, microcrystals and even granular precipitates, suitable crystals have been obtained. In the two above-mentioned case studies, The optimizer plate was key in the production of co-crystals between a protein and 3 different ligands and well defined 3D crystals (2.8 Å on home source) of an important protein target. For each of the case study, results were obtained in a single microplate, prepared in minutes. Every experiment led to a variety of results from clear drop to heavy precipitate, showing the influence of the optimization solution mix on the protein solubility. The use of a variety of salts as optimizers highlights the differences between the cation (sodium, potassium, ammonium, magnesium, calcium and lithium) and the anion (formate, malonate, chloride, acetate, fluoride, nitrate, thiocyanate, etc) part of salts. Other Optimizer plates using Pegs, organics and other chemicals as co-precipitant can also be used.

This new pre-filled optimizer plate represents a promising alternative to a standard grid approach when performing optimization. It is easier and faster to setup and bring a lot of information on effect of salt concentration, buffers, and additives on crystallization of a particular protein. Effective 96 optimization conditions can be prepared in less than 10 minutes.

The optimization strategy described herein can be applied as soon as crystal forms appear in a drop. It is a faster and easier method than those now in existence. The simple addition of someone's hit condition to each of the 96 chemicals in the pre-filled plate makes this optimization technique rapid and simple. Since the chemical compositions of these micro-plates are so different, the results are actually a 2^{nd} level of screening based on a partly successful 1^{st} level initial screening. By using this simple method, it is now possible to rapidly see if a “mild change” in the chemical environment will be beneficial or not, compare to a very “soft change” brought in by a factorial approach of optimization, as is currently being done.

In a successful crystallization strategic plan, two (2) aspects of crystallization, i.e. an initial screening and an optimization, must be integrated. To maximize the interaction of the two, results from one technique must be easily processed and bring success in the following one. In this case, while working with 6 specific proteins, this interaction between initial screening and optimization was tested on 54 different crystallization results. Once the optimization technique was selected, major improvement was seen in 85% of the cases (46/54). By combining an initial screening plan (large chemical variety with 2 concentration of precipitants) and a solid optimization procedure like the Optimizer plate, it is now possible to react rapidly during crystal growth and get the sought after success, i.e. diffraction-quality crystals.

Of course, one skilled in the art will readily appreciate that the present invention as now disclosed can also be used as a transfer plate, and not only a crystallization plate. For example, plates containing in each well sufficient optimizing solutions (crystallization media) for a number of assays could be used and sold. instead of 10 μl be put in each well, a plate that would have 250 μl per well could thus be used for 10 assays (assuming there is no loss or evaporation of the media). Furthermore, the person skilled in the art will appreciate that correction of concentration of the reagents (for example the hit solution) may be desired.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A multi-well plate comprising a plurality of wells, each well having therein a different crystallization media, each crystallization media varying according to at least two different parameters, a first parameter having at least one condition, and a second parameter having at least two different conditions, whereby said multi-well plate allows facilitating optimization of crystallization conditions of a substrate.

2. The plate of claim 1, wherein said parameters are selected from the group consisting of a buffer, pH of said crystallization media, salt, concentration of said salt, temperature of said crystallization media, additive, concentration of said additive, co-crystallization compound, concentration of said co-crystallization compound, alcohol, concentration of said alcohol, polymer, concentration of said polymer.

3. The plate of claim 2, wherein one of said parameters is the buffer.

4. The plate of claim 3, wherein each condition of said buffer parameter is represented by a predetermined buffer.

5. The plate of claim 4, wherein said predetermined buffer is selected from the group consisting of Tris, Tris HCI, HEPES, Sodium HEPES, Imidazole, Sodium Citrate, Sodium Cacodylate and Sodium Acetate.

6. The plate of claim 2, wherein one of said parameters is the pH of said crystallization media.

7. The plate of claim 6, wherein each condition of the pH is represented by a predetermined value of pH.

8. The plate of claim 2, wherein one of said parameters is salt.

9. The plate of claim 8, wherein each condition of the salt is represented by a predetermined salt.

10. The plate of claim 9, wherein said predetermined salt comprises an inorganic or an organic anion, and an organic cation.

11. The plate of claim 9, wherein said predetermined salt comprises an organic anion, and an inorganic or an organic cation.

12. The plate of claim 9, wherein said predetermined salt comprises a cation selected from the group consisting of sodium, potassium, ammonium, magnesium, calcium, and lithium.

13. The plate of claim 9 or 12, wherein said predetermined salt comprises a anion selected from the group consisting of formate, malonate, chloride, acetate, fluoride, bromide, nitrate and thiocyanate.

14. The plate of claim 2, wherein one of said parameters is the concentration of said salt.

15. The plate of claim 14, wherein each condition of the salt concentration is represented by a predetermined concentration value of said salt.

16. The plate of claim 2, wherein one of said parameters is the temperature of said crystallization media.

17. The plate of claim 16, wherein each condition of the temperature media is represented by a predetermined temperature.

18. The plate of claim 2, wherein one of said parameters is the additive.

19. The plate of claim 18, wherein each condition of the additive is represented by a predetermined additive.

20. The plate of claim 19, wherein said predetermined additive is selected from the group consisting of reducing agents, metal ions, inhibitors and a detergent.

21. The plate of claim 2, wherein one of said parameters is the concentration of said additive.

22. The plate of claim 21, wherein each condition of the additive concentration is represented by a predetermined concentration value of said additive.

23. The plate of claim 2, wherein one of said parameters is the ligand.

24. The plate of claim 23, wherein each condition of the ligand is represented by a predetermined ligand.

25. The plate of claim 24, wherein said predetermined ligand is selected from the group consisting of ATP, ADP, NAD, NADH, NADP, and NADPH.

26. The plate of claim 2, wherein one of said parameters is the concentration of said ligand.

27. The plate of claim 26, wherein each condition of the ligand concentration is represented by a predetermined concentration value of said ligand.

28. The plate of claim 2, wherein one of said parameters is the alcohol.

29. The plate of claim 28, wherein each condition of the alcohol is represented by a predetermined alcohol.

30. The plate of claim 29, wherein said predetermined alcohol is selected from the group consisting of methanol, ethanol, propanol isopropanol, methylpentanediol, hexanediol, and ethylene glycol.

31. The plate of claim 2, wherein one of said parameters is the concentration of said alcohol.

32. The plate of claim 31, wherein each condition of the alcohol concentration is represented by a predetermined concentration value of said alcohol.

33. The plate of claim 2, wherein one of said parameters is the polymer.

34. The plate of claim 33, wherein each condition of the polymer is represented by a predetermined polymer.

35. The plate of claim 34, wherein said predetermined polymer is selected from the group consisting of PEG, polyethyleneimine and Jeffamine M-600.

36. The plate of claim 2, wherein one of said parameters is the concentration of said polymer.

37. The plate of claim 36, wherein each condition of the polymer concentration is represented by a predetermined concentration value of said polymer.

38. The plate of any one of claims 2 to 37, wherein said crystallization media varies according to three different parameters, a first parameter having at least one condition, and a second parameter having at least two different conditions, and a third parameter having at least one condition.

39. The plate of any one of claims 2 to 37, wherein said crystallization media varies according to three different parameters, a first parameter having at least one condition, and a second parameter having at least two different conditions, and a third parameter having at least two different conditions.

40. The plate of claim 38 or 39, wherein said first parameter is the additive, said second parameter is the concentration of said additive, and said third parameter is the pH of said crystallization media.

41. The plate of claim 38 or 39, wherein said first parameter is the salt, said second parameter is the concentration of said salt, and said third parameter is the pH of said crystallization media.

42. The plate of any one of claims 1 to 41, comprising 3, 6, 24, 96, 192, 384, 768 or 1536 wells.

43. The plate of any one of claims 1 to 41, comprising 96 wells.

44. The plate of claim 38 or 39, comprising 96 wells, said first parameter being the salt and the conditions of said first parameter being 16 different salts, said second parameter being the salt concentration and the conditions of said second parameter being 2 different concentrations, and said third parameter being the pH and the conditions of said third parameter are 3 different pH values.

45. The plate of any one of claims 1 to 44 wherein said crystallization media is a solution.

46. The plate of any one of claims 1 to 44, wherein said crystallization media is a gel.

47. The plate of any one of claims 1 to 46, further comprising a cover disposed on said wells to seal said wells.

48. The plate of any one of claims 1 to 46, wherein said plate is of the hanging-drop crystallization type of plate, said plate further comprising a cover for sealing said wells.

49. The plate of any one of claims 1 to 46, wherein said plate is of the sitting-drop crystallization type of plate.

50. The plate of any one of claims 49, wherein each well comprises a crystallization media reservoir adjacent to a substrate well.

51. The plate of any one of claims 1 to 50, wherein said substrate is a protein.

52. The plate of any one of claims 1 to 51, wherein each well comprises at least 1 μL of said crystallization media.

53. The plate of any one of claims 1 to 51, wherein each well comprises about 5 to about 500 μL of said crystallization media.

54. The plate of any one of claims 1 to 51, wherein each well comprises about 10 μL of said crystallization media.

55. The plate of any one of claims 1 to 46, further comprising a cover disposed on said wells to seal said wells, and wherein each well comprises a crystallization media reservoir adjacent to a substrate well.

56. A method for optimizing crystallization conditions for a substrate comprising the step of adding said substrate into each well of a plate as defined in any one of claims 1 to 55.

57. The method of claim 56, wherein said method further comprises adding a hit solution for said substrate in each well before adding said substrate in each well.

58. A method for optimizing crystallization conditions for a substrate comprising the step of contacting said substrate with a hit solution for said substrate, and said crystallization media into each well of a plate as defined in any one of claims 1 to 55.

59. A method for optimizing crystallization of a substrate comprising:
a) determining a hit solution for said substrate by screening different solutions; and
b) adding said hit solution determined in step a) into each media reservoir of a plate as defined in claim 50 so as to obtain a mixture;
c) adding a substrate into substrate wells;
d) transferring a desired volume of said mixture from each media reservoir to the substrate wells; and
e) sealing said substrate wells and media reservoir and allowing for crystallization of the substrate.

60. A method for optimizing crystallization conditions for a substrate comprising:
a) determining a hit solution for said substrate by screening different solutions; and
b) adding said hit solution into a media reservoir of a plate as defined in claim 55 so as to obtain a mixture of said crystallization media and said hit solution;
c) adding said substrate in the substrate well;
d) adding the mixture obtained at step b) in the substrate well; and
e) sealing the media reservoir and the adjacent substrate well with said cover, and allowing crystallization of the substrate.

61. A method for optimizing crystallization conditions for a substrate comprising:
a) determining a hit solution for said substrate by screening different solutions; and
b) transferring from the wells of a plate as defined in claim 1 to the wells of a crystallization plate the crystallization media;
c) adding said hit solution to the crystallization media in the wells of the crystallization plate so as to obtain a mixture of said crystallization media and said hit solution;
d) adding said substrate in the well of the crystallization well; and
e) sealing the wells of the crystallization plate with a cover, and allowing crystallization of the substrate.
